(19) United States
(12) Patent Application Publication
(10) Pub. No.: US 2019/0351123 A1
(43) Pub. Date: Nov. 21, 2019

(54) MEDICAL FLUID DELIVERY SYSTEM WITH SELF-REGISTERING FLUID CONNECTOR

(71) Applicants: Baxter International Inc., Deerfield, IL (US); Baxter Healthcare SA, Glattpark (Opfikon) (CH)

(72) Inventors: John S. Norman, Gurnee, IL (US); Edward S. Szpara, St. Charles, IL (US); James D. Ascenzo, Glen Ellyn, IL (US)

(21) Appl. No.: 16/413,284

(22) Filed: May 15, 2019

Related U.S. Application Data
(60) Provisional application No. 62/672,813, filed on May 17, 2018.

(51) Int. Cl. A61M 1/28; A61M 1/16

(52) U.S. Cl. CPC A61M 1/287; A61M 1/1696; A61M 1/1668; A61M 1/3621; A61M 2205/128; A61M 1/1601; A61M 2205/121

(57) ABSTRACT

A medical fluid connector includes a first port; a second port; a pivot located between the ports; a guide wall located between the first port and the pivot and positioned to reside adjacent to a first side of a wall of a structure supporting the connector when inserted into the structure; and a guide member located between the second port and the pivot and including a member wall positioned to reside adjacent to a second side of the wall of the structure supporting the connector when inserted into the structure, the guide member further including at least one member flange extending from the member wall, the member flange sized so as to provide room for a lid of the structure to close onto the member flange and rotate the connector about the pivot into a desired operating position. A dialysis system, disposable and method using the connector are also provided.

### RELATED APPLICATIONS

This application claims the benefit of and priority to U.S. Provisional Patent Application No. 62/672,813 filed May 17, 2018, entitled “MEDICAL FLUID DELIVERY SYSTEM WITH SELF-REGISTERING FLUID CONNECTOR,” which is incorporated herein by reference in its entirety.

### BACKGROUND

The present disclosure relates generally to medical fluid devices. More specifically, the present disclosure relates to medical fluid devices that mix fluid online for treatment or that receive fluid mixed online for treatment.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney function is critical to many people because the treatment is life saving.

One type of kidney failure therapy is hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD (HF, HDF) treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products than a patient receiving less frequent but perhaps longer treatments. A patient receiving more frequent treatments does not experience as much of a down cycle as does an in-center patient, who has built-up two or three day's worth of toxins prior to a treatment. In certain areas, the closest dialysis center may be many miles from the patient's home, causing door-to-door treatment time to consume a large portion of the day. HHD can take place overnight or during the day while the patient relaxes, works or is otherwise productive.

Another type of kidney failure therapy is peritoneal dialysis (“PD”), which infuses a dialysis solution, also called dialysis fluid, into a patient's peritoneal cavity via a catheter. The dialysis fluid contacts the peritoneal membrane of the peritoneal cavity. Waste, toxins and excess water pass from the patient's bloodstream, through the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used or spent dialysis fluid to drain from the peritoneal cavity. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal cavity, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysis fluid and to a fluid drain. APD machines pump fresh dialysis fluid from a dialysis fluid source, through the catheter and into the patient's peritoneal cavity. APD machines also allow for the dialysis fluid to dwell within the cavity and for the transfer of waste, toxins and excess water to take place. The source may include multiple sterile dialysis fluid solution bags.

APD machines pump used or spent dialysate from the peritoneal cavity, through the catheter, and to the drain. As with the manual process, several drain, fill and dwell cycles occur during dialysis. A “last fill” may occur at the end of the APD treatment. The fluid may remain in the peritoneal cavity of the patient until the start of the next treatment, or may be manually emptied at some point during the day.

In any of the above modalities using an automated machine, treatment fluid may be prepared online or at the point of use, e.g., before and/or during the treatment. It is important that the treatment fluid, e.g., dialysis fluid be mixed properly and homogeneously for treatment. A need exists for improved mixing accordingly.

### SUMMARY

The examples described herein disclose automated systems and methods applicable, for example, to fluid delivery for: peritoneal dialysis (“PD”), plasmapheresis, hemodialysis (“HD”), hemofiltration (“HF”) hemodiafiltration (“HDF”), continuous renal replacement therapy (“CRRT”), apheresis, autotransfusion, hemofiltration for sepsis, and extracorporeal membrane oxygenation (“ECMO”) treatments. The systems and methods described herein are applicable to any medical fluid delivery system in which the treatment fluid may be made online or at the point of use, e.g., just before and/or during treatment. These modalities may be referred to collectively or generally individually herein as medical fluid delivery system(s).

Moreover, each of the systems and methods described herein may be used with clinical or home-based treatments. For example, the present systems and methods may be employed in in-center PD, HD, HF or HDF machines, which run throughout the day. Alternatively, the present systems and methods may be used with home PD, HD, HF or HDF machines, which are operated generally at the patient's convenience.

In one embodiment, a peritoneal dialysis system and method are provided having point of use dialysis fluid production. The system includes a cycler and a water purifier. The cycler includes a control unit having at least one processor and at least one memory. The cycler may further include a wired or wireless transceiver for sending information to and receiving information from the water purifier. The water purifier may also include a control unit having at least one processor and at least one memory and a wired or wireless transceiver for sending information to and receiving information from the control unit of the cycler.

The cycler includes equipment programmed via its control unit to prepare fresh dialysis solution at the point of use, pump the freshly prepared dialysis fluid to a patient, allow the dialysis fluid to dwell within the patient, then pump used dialysis fluid to a drain. The cycler in one embodiment includes a heater under control of the control unit for heating the dialysis fluid as it is being mixed in one embodiment. The heater may for example be located at the top of a housing of the cycler, e.g., beneath a heating lid.

The cycler (and the water purifier in one embodiment) operates with a disposable set. The disposable set in one embodiment includes a disposable pumping cassette, which may include a planar rigid plastic piece covered on one or both sides by a flexible membrane, forming fluid pumping and valving chambers. The fluid pump chambers may operate with pneumatic pump chambers of the cycler, while fluid valve chambers operate with the pneumatic valve chambers of the cycler.

The disposable set may include (i) a patient line that extends from the cassette to a patient line connector, (ii) a drain line that extends from the cassette to a drain line connector (which may in turn connect removeably to the water purifier), (iii) a heater/mixing line that extends from the pumping cassette to a heater/mixing bag of the present disclosure, (iv) an upstream water line segment that extends from the water purifier to a water inlet of a water accumulator and a downstream water line segment that extends from a water outlet of the water accumulator to the cassette, (v) a last bag or sample line that extends from the cassette to a premixed last fill bag of dialysis fluid or to a sample bag or other sample collecting container, (vi) a first, e.g., glucose, concentrate line extending from the cassette to a first, e.g., glucose, concentrate container, and/or (vii) a second, e.g., buffer, concentrate line that extends from the cassette to a second, e.g., buffer, concentrate container.

The heater/mixing bag or container is provided with a self-registering connector. The connector includes a first port sealed to the heater/mixing container, e.g., is mechanically sealed, heat sealed, solvent bonded and/or ultrasonically welded to the heater/mixing container. The connector includes a second port sealed to the heater/mixing line extending from the container or bag of the present disclosure, e.g., sealed via any of the methods for sealing to the first port. The connector includes a pivot located between the first and second ports. The pivot is in one embodiment rectangular with rounded top and bottom surfaces. The pivot rotates within a semicircular flange formed with or provided by a sidewall of a heater/mixing tray formed with the housing of the dialysis machine or cycler.

The connector includes a guide wall located between the first or heater/mixing bag port and the pivot. The guide wall is positioned on the connector so as to reside just inside and in one embodiment contact an inside surface of the sidewall of the heater/mixing tray when the connector is placed into a slot formed in the sidewall for mounting the connector. The guide wall is formed in the shape of a polygon and is sized sufficiently to help prevent the connector from being rotated during treatment (e.g., via fluid pressure fluctuation or inadvertent bumping) about an axis extending through the sidewall when the connector is inserted into the slot in the sidewall. The guide wall is in one embodiment at least substantially flat.

The connector also includes a guide member located between the second or heater/mixing line port and the pivot. The guide member includes a member wall positioned on the connector so as to reside just outside and in one embodiment contact an outside surface of the sidewall of the heater/mixing tray when the connector is placed into the slot formed in the sidewall for mounting the connector. The member wall is formed in the shape of a polygon (e.g., the same polygonal shape of the guide wall) and is sized sufficiently to help prevent the connector from being rotated during treatment (e.g., via fluid pressure fluctuation or inadvertent bumping) about an axis extending through the sidewall when the connector is inserted into the slot in the sidewall. The member wall may be sized at least substantially the same as the guide wall but may be longer in certain dimensions that the guide wall.

The guide member includes multiple member flanges that extend from the member wall in the same direction as the second or heater/mixing line port. The member flanges may be connected and molded as a single piece with the guide wall to form the guide member. In one embodiment, five member flanges are provided to form an open-ended, trapezoidal box, which is strong and readily capable of being contacted and rotated via a lid of the housing covering the heater/mixing tray to self-register the connector.

In one embodiment, no flange member is provided along a bottom the member wall of the guide member, so that the pivot, which may extend at least part of the way through the bottom of the guide member, may be placed into the semi-circular flange formed with or provided by the sidewall of the heater/mixing tray. The bottom of the member wall of the guide member may however be provided with first and second notches that serve to provide ends of travel. One of the notches is positioned and arranged to abut a first horizontal edge surface of the semi-circular flange when the heater/mixing container is placed in the heater/mixing tray and the connector is placed in the slot of the sidewall, prior to rotation of the connector. Another one of the notches is positioned and arranged to abut a second horizontal edge surface of the semi-circular flange at the end of travel when the connector is fully rotated via the closing of the heater lid to place the connector in a desired position for treatment.

The desired position for treatment is in one embodiment a substantially horizontal position, which helps to prevent different fluids to be mixed in the heater/mixing container from separating upon entry into the heater/mixing container. For example, if the heater/mixing bag port of the connector is instead pointed upward from horizontal, the heavier concentrate may tend to separate from the purified water upon entering the heater/mixing container.

The member flanges are made wide enough to account for any misalignment between the heater lid and the sidewalls of the heater/mixing tray when the lid closes onto the tray to ensure that the lid contacts one or more of the member flanges. When the lid is closed, the lid contacts the top of the connector at one ore more of the member flanges (e.g., at an intersection of two member flanges) and moves or rotates the connector into the desired operating position. The lid while closed during operation maintains contact with the connector to prevent the connector from straying from the desired operating position, e.g., via fluid pressure fluctuations or inadvertent bumping of the dialysis cycler. The housing of the machine or cycler may also be formed with a ledge that is contacted by one of the member flanges when the connector is rotated fully into the desired position for treatment. Here, the connector is trapped between the lid and the ledge during treatment.

The connector and in particular the guide member of the connector is configured such that if the patient or caregiver loads the heater/mixing container upside down into the heater/mixing tray, the connector is prevented from sliding into the slot in the heater/mixing tray sidewall, and the lid is prevented from closing fully. Such tactile and/or visual feedback may inform the patient or caregiver to reload the heater/mixing container properly into the heater/mixing tray. It is also contemplated to provide a switch or sensor that senses when the heater lid is closed fully, and to program a control unit of the machine or cycler such that if the patient or caregiver attempts to begin treatment when the lid is not full closed, the control unit causes the user interface of the machine or cycler to display and/or sound an alarm and a message to check the loading of the heater/mixing container in the heater/mixing tray.

In light of the disclosure herein and without limiting the disclosure in any way, in a first aspect of the present disclosure, which may be combined with any other aspect listed herein unless specified otherwise, a medical fluid connector includes: a first port; a second port; a pivot located between the first port and the second port; a guide wall located between the first port and the pivot, the guide wall positioned to reside adjacent to a first side of a wall of a structure supporting the connector when inserted into the structure; and a guide member located between the second port and the pivot, the guide member including a member wall positioned to reside adjacent to a second side of the wall of the structure supporting the connector when inserted into the structure, the guide member further including at least one member flange extending from the member wall, the at least one member flange sized so as to provide room for a lid of the structure to close onto the at least one member flange and rotate the connector about the pivot into a desired operating position.

In a second aspect of the present disclosure, which may be combined with any other aspect listed herein unless specified otherwise, wherein the guide wall and the member wall are at least partially shaped the same.

In a third aspect of the present disclosure, which may be combined with any other aspect listed herein unless specified otherwise, the guide wall and the member wall are at least partially sized the same.

In a fourth aspect of the present disclosure, which may be combined with any other aspect listed herein unless specified otherwise, the member wall is formed as a polygon, and wherein plural member flanges extend around a perimeter of the polygon, leaving an opening without a member flange at a bottom of the member wall, wherein the bottom of the member wall is configured to be received by the structure supporting the connector.

In a fifth aspect of the present disclosure, which may be combined with any other aspect listed herein unless specified otherwise, the plural flanges and the member wall are molded together to form the connector.

In a sixth aspect of the present disclosure, which may be combined with any other aspect listed herein unless specified otherwise, a bottom of the member wall forms a notch, which when the connector is rotated into the desired operating position, the notch abuts against a portion of the structure.

In a seventh aspect of the present disclosure, which may be combined with the sixth aspect in combination any other aspect listed herein unless specified otherwise, the notch is a first notch, and wherein the bottom of the member wall forms a second notch that contacts a second portion of the structure prior to the connector being rotated.

In an eighth aspect of the present disclosure, which may be combined with any other aspect listed herein unless specified otherwise, the pivot is formed in a rectangular shape, and wherein at least one of the sides is rounded for rotation against the structure supporting the connector.

In a ninth aspect of the present disclosure, which may be combined with any other aspect listed herein unless specified otherwise, a bottom of the pivot extends through at least a portion of the guide member.

In a tenth aspect of the present disclosure, which may be combined with any other aspect listed herein unless specified otherwise, a dialysis system includes: a source of water made suitable for a dialysis treatment; at least one concentrate for mixing with the water from the source; a housing holding a dialysis fluid pump and forming a tray and a lid covering the tray; and a disposable set operable with the dialysis fluid pump and in fluid communication with the source of water and the at least one concentrate, the disposable set including a container configured to accept the water and the at least one concentrate pumped by the dialysis fluid pump via a connector to mix for the dialysis treatment, wherein the connector is positioned and arranged such that when the container is placed onto the tray, the lid is able to close onto and move the connector into a desired operating position.

In an eleventh aspect of the present disclosure, which may be combined with the tenth aspect in combination any other aspect listed herein unless specified otherwise, the disposable set incudes a pumping cassette operable with the dialysis fluid pump, and wherein the connector includes a first port and a second port, the first port sealed to the container, and the second port sealed to a line leading from the connector to the pumping cassette.

In a twelfth aspect of the present disclosure, which may be combined with the tenth aspect in combination any other aspect listed herein unless specified otherwise, the lid is hinged to the housing and closes rotatingly onto the connector.

In a thirteenth aspect of the present disclosure, which may be combined with the tenth aspect in combination any other aspect listed herein unless specified otherwise, the connector is configured to be rotated into the desired operating position when moved by the lid.

In a fourteenth aspect of the present disclosure, which may be combined with the tenth aspect in combination any other aspect listed herein unless specified otherwise, the tray is a heater tray and the container is sized to enable the heater tray to heat the water and the at least one concentrate as they mix in the container.

In a fifteenth aspect of the present disclosure, which may be combined with the tenth aspect in combination any other aspect listed herein unless specified otherwise, the tray defines a slot and includes a flange located beneath the slot, the flange including a surface, and wherein the connector is configured such that when moved into the desired operating position, the connector abuts against the surface.

In a sixteenth aspect of the present disclosure, which may be combined with the fifteenth aspect in combination any other aspect listed herein unless specified otherwise, the surface is a first surface, wherein the flange includes a second surface, and wherein the connector is configured to contact the second surface prior to the connector being moved by the lid.

In a seventeenth aspect of the present disclosure, which may be combined with the fifteenth aspect in combination any other aspect listed herein unless specified otherwise, the flange is a semicircular flange, and wherein the connector is configured to be rotated within the semicircular flange when the connector is moved by the lid.

In an eighteenth aspect of the present disclosure, which may be combined with the tenth aspect in combination any other aspect listed herein unless specified otherwise, the housing further defines a ledge, and wherein the connector is sized and arranged to abut against the ledge when moved by the lid into the desired operating position.

In a nineteenth aspect of the present disclosure, which may be combined with any other aspect listed herein unless specified otherwise, a disposable set includes: a pumping cassette; a heater/mixing container; a line extending from the pumping cassette to the heater mixing container; and a connector including a first port sealed to the heater mixing container, a second port sealed to the line extending from the pumping cassette, a pivot located between the first port and the second port, and a guide member located between the second port and the pivot, the guide member including a member wall positioned to reside adjacent to a side of a wall of a structure supporting the connector when inserted into the structure, the guide member further including at least one member flange extending from the member wall, the at least one member flange positioned and arranged to provide room for a lid of the structure to close onto the member flange and rotate the connector about the pivot into a desired operating position.

In a twentieth aspect of the present disclosure, which may be combined with the nineteenth aspect in combination any other aspect listed herein unless specified otherwise, the disposable set includes a guide wall located between the first port and the pivot, the guide wall positioned to reside adjacent to an opposing side of the wall of the structure supporting the connector when inserted into the structure.

In a twenty-first aspect of the present disclosure, which may be combined with the nineteenth aspect in combination any other aspect listed herein unless specified otherwise, a bottom of the pivot extends through at least a portion of the guide member.

In a twenty-second aspect of the present disclosure, which may be combined with any other aspect listed herein unless specified otherwise, a dialysis method includes: providing a tray having a slot; coupling a lid adjacent to the tray so that the lid may be opened and closed relative to the tray; providing a fluid container having a connector sealed to the fluid container; sizing the slot and a portion of the connector to fit into the slot when the container is placed onto the tray; and configuring the connector such that when the lid is closed relative to the tray, the lid contacts the connector and moves the connector into a desired position for treatment.

In a twenty-third aspect of the present disclosure, which may be combined with the twenty-second aspect in combination any other aspect listed herein unless specified otherwise, coupling the lid includes hinging the lid to the tray.

In a twenty-fourth aspect of the present disclosure, which may be combined with the twenty-second aspect in combination any other aspect listed herein unless specified otherwise, moving the connector includes rotating the connector.

In a twenty-fifth aspect of the present disclosure, which may be combined with the twenty-second aspect in combination any other aspect listed herein unless specified otherwise, the dialysis method includes configuring the slot, the connector and the lid so the lid continues to hold the connector in the desired position during treatment.

In a twenty-sixth aspect of the present disclosure, which may be combined with the twenty-second aspect in combination any other aspect listed herein unless specified otherwise, the dialysis method includes configuring the connector and the slot such that a portion of the connector dead ends against a portion of the slot when the connector is in the desired position during treatment.

In a twenty-seventh aspect of the present disclosure, which may be combined with the twenty-second aspect in combination any other aspect listed herein unless specified otherwise, the dialysis method includes providing the tray as part of a housing of a dialysis machine, and configuring the connector and the housing such that a portion of the connector dead ends against a portion of the housing when the connector is in the desired position during treatment.

In a twenty-eighth aspect of the present disclosure, any of the structure, functionality and alternatives disclosed in connection with FIGS. 1 to 7B may be combined with any of the other structure, functionality and alternatives disclosed in connection with FIGS. 1 to 7B.

In light of the present disclosure and the above aspects, it is therefore an advantage of the present disclosure to provide an improved medical fluid delivery system.

It is another advantage of the present disclosure to provide an improved medical fluid delivery system that prepares treatment fluid online or at the point of use.

It is a further advantage of the present disclosure to provide an improved mixing structure and methodology for a medical fluid delivery system that prepares treatment fluid online or at the point of use.

The advantages discussed herein may be found in one, or some, and perhaps not all of the embodiments disclosed herein. Additional features and advantages are described herein, and will be apparent from, the following Detailed Description and the figures.

### BRIEF DESCRIPTION OF THE FIGURES

FIG. 1 is a front elevation view of one embodiment of a medical fluid delivery system having point of use dialysis fluid production of the present disclosure.

FIG. 2 is an elevation view of one embodiment of a disposable set used with the system illustrated in FIG. 1.

FIGS. 3A and 3B illustrate various views of one embodiment of a heater/mixing tray operable with a heater/mixing bag having a self-registering connector of the present disclosure.

FIG. 4A is an enlarged side elevation view of one embodiment of the self-registering connector illustrated in FIG. 3A.

FIG. 4B is an outside-in end elevation view of the self-registering connector illustrated in FIG. 4A.

FIG. 4C is an inside-out end elevation view of the self-registering connector illustrated in FIG. 4A.

FIG. 4D is a top plan view of the self-registering connector illustrated in FIG. 4A.

FIG. 4E is a bottom plan view of the self-registering connector illustrated in FIG. 4A.

FIG. 4F is an sectioned outside-in elevation view taken along line 4F-4F in FIG. 4D.

FIG. 4G is a sectioned side elevation view taken along line IVG-IVG in FIG. 4C.

FIG. 4H is a sectioned bottom view taken along line IVH-IVH in FIG. 4A.

FIG. 5A is a perspective outside-in view of one embodiment of a lid of a medical fluid device of the present disclosure as it is just about to close onto an embodiment of the self-registering connector, which is in its initially placed, non-rotated position.

FIG. 5B is a perspective outside-in view of the lid of FIG. 5A after it has closed fully onto the heater/mixing pan of FIGS. 3A and 3B, which causes the self-registering connector to be rotated into its operational position.

FIG. 5C is a perspective outside-in view of the lid of FIGS. 5A and 5B being prevented from closing fully when the heating/mixing bag has been loaded upside down mistakenly.

FIG. 6A is a side elevation inside-out view of one embodiment of a lid of a medical fluid device of the present disclosure as it is just about to close onto an embodiment of the self-registering connector, which is in its initially placed, non-rotated position.

FIG. 6B is a side elevation inside-out view of the lid of FIG. 6A after it has closed fully onto the heater/mixing pan of FIGS. 3A and 3B, which causes the self-registering connector to be rotated into its operational position.

FIGS. 7A and 7B are the same views as FIGS. 6A and 6B, respectively, showing the self-registering connector in hidden line behind the sidewall of the heater/mixing tray.

### DETAILED DESCRIPTION

### System Overview

The examples described herein are applicable to any medical fluid therapy system that delivers a medical fluid that may be mixed at the point of use, prior to and/or during treatment, such as dialysis fluid, substitution fluid, or an intravenous drug. The examples are particularly well suited for kidney failure therapies, such as all forms of peritoneal dialysis (“PD”), hemodialysis (“HD”), hemofiltration (“HF”), hemodiafiltration (“HDF”) and continuous renal replacement therapies (“CRRT”), referred to herein collectively or generally individually as renal failure therapy. Moreover, the machines described herein may be used in clinical or home settings. For example, the machines and associated methods may be employed in an in-center PD or HD machine, which runs virtually continuously throughout the day. Alternatively, the machine and methods may be used in a home PD or HD machine, which can for example be run at night while the patient is sleeping. The machines and methods discussed herein are also applicable to medical delivery applications. The following examples will be described in the setting of a peritoneal dialysis system having point of use dialysis fluid production but may instead be used to make point of use treatment fluid for any of the above modalities.

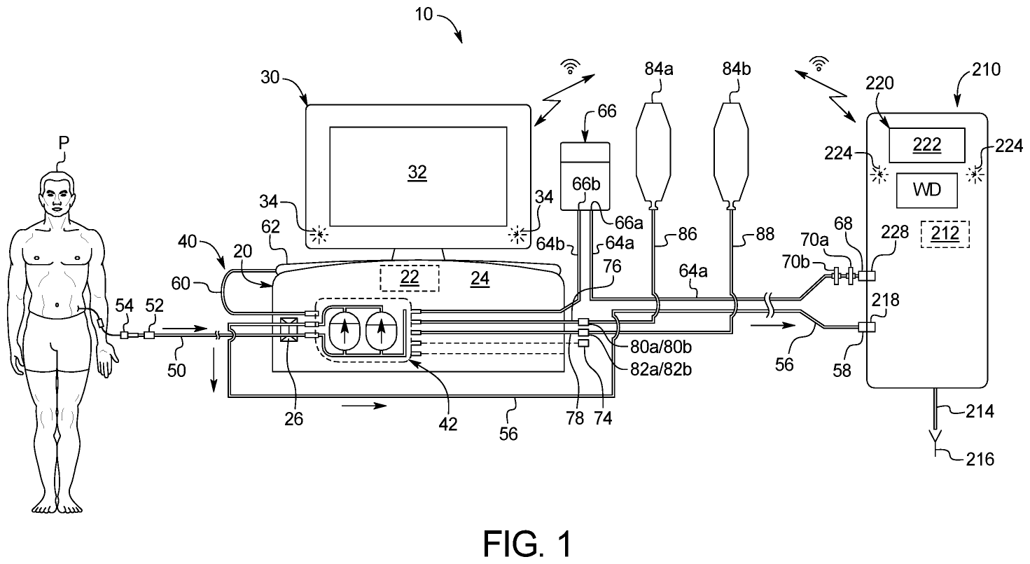

Referring now to the drawings and in particular to FIG. 1, one embodiment of a peritoneal dialysis system having point of use dialysis fluid production of the present disclosure is illustrated by system 10. System 10 includes a cycler 20 and a water purifier 210. Suitable cyclers for cycler 20 include, e.g., the Amia® or HomeChoice® cycler marketed by Baxter International Inc., with the understanding that those cyclers are provided with updated programming to perform and use the point of use dialysis fluid produced according to system 10. To this end, cycler 20 includes a control unit 22 having at least one processor and at least one memory. Control unit 22 further incudes a wired or wireless transceiver for sending information to and receiving information from a water purifier 210. Water purifier 210 also includes a control unit 212 having at least one processor and at least one memory. Control unit 212 further incudes a wired or wireless transceiver for sending information to and receiving information from control unit 22 of cycler 20. Wired communication may be via Ethernet connection, for example. Wireless communication may be performed via any of Bluetooth™, WiFi™, Zigbee®, Z-Wave®, wireless Universal Serial Bus (“USB”), or infrared protocols, or via any other suitable wireless communication technology.

Cycler 20 includes a housing 24, which holds equipment programmed via control unit 22 to prepare fresh dialysis solution at the point of use, pump the freshly prepared dialysis fluid to patient P, allow the dialysis fluid to dwell within patient P, then pump used dialysis fluid to a drain. In the illustrated embodiment, water purifier 210 includes a drain line 214 leading to a drain 216, which can be a house drain or a drain container. The equipment programmed via control unit 22 to prepare fresh dialysis solution at the point of use in an embodiment includes equipment for a pneumatic pumping system, including but not limited to (i) one or more positive pressure reservoir, (ii) one or more negative pressure reservoir, (iii) a compressor and a vacuum pump each under control of control unit 22, or a single pump creating both positive and negative pressure under control of control unit 22, to provide positive and negative pressure to be stored at the one or more positive and negative pressure reservoirs, (iv) plural pneumatic valve chambers for delivering positive and negative pressure to plural fluid valve chambers, (v) plural pneumatic pump chambers for delivering positive and negative pressure to plural fluid pump chambers, (vi) plural electrically actuated on/off pneumatic solenoid valves under control of control unit 22 located between the plural pneumatic valve chambers and the plural fluid valve chambers, (vii) plural electrically actuated variable orifice pneumatic valves under control of control unit 22 located between the plural pneumatic pump chambers and the plural fluid pump chambers, (viii) a heater under control of control unit 22 for heating the dialysis fluid as it is being mixed in one embodiment, and (ix) an occluder 26 under control of control unit 22 for closing the patient and drain lines in alarm and other situations.

In one embodiment, the plural pneumatic valve chambers and the plural pneumatic pump chambers are located on a front face or surface of housing 24 of cycler 20. The heater is located inside housing 24 and in an embodiment includes heating coils that contact a heating pan or tray, which is located at the top of housing 24, beneath a heating lid (not seen in FIG. 1).

Cycler 20 in the illustrated embodiment includes a user interface 30. Control unit 22 in an embodiment includes a video controller, which may have its own processing and memory for interacting with primary control processing and memory of control unit 22. User interface 30 includes a video monitor 32, which may operate with a touch screen overlay placed onto video monitor 32 for inputting commands via user interface 30 into control unit 22. User interface 30 may also include one or more electromechanical input device, such as a membrane switch or other button. Control unit 22 may further include an audio controller for playing sound files, such as voice activation commands, at one or more speaker 34.

Water purifier 210 in the illustrated embodiment also includes a user interface 220. Control unit 212 of water purifier 210 in an embodiment includes a video controller, which may have its own processing and memory for interacting with primary control processing and memory of control unit 212. User interface 220 includes a video monitor 222, which may likewise operate with a touch screen overlay placed onto video monitor 222 for inputting commands into control unit 212. User interface 220 may also include one or more electromechanical input device, such as a membrane switch or other button. Control unit 212 may further include an audio controller for playing sound files, such as alarm or alert sounds, at one or more speaker 224 of water purifier 210.

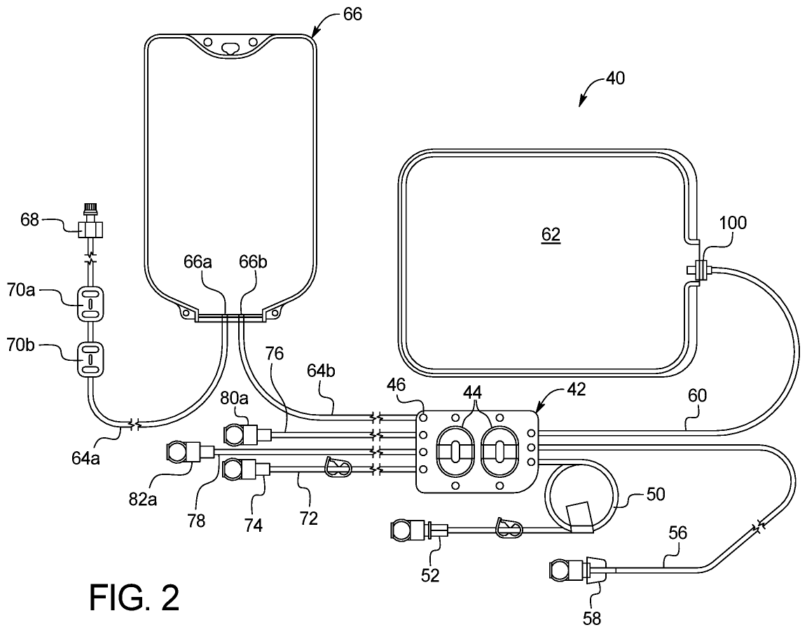

Referring additionally to FIG. 2, one embodiment of disposable set 40 is illustrated. Disposable set 40 is also illustrated in FIG. 1, mated to cycler 20 to move fluid within the disposable set 40, e.g., to mix dialysis fluid as discussed herein. Disposable set 40 in the illustrated embodiment includes a disposable cassette 42, which may include a planar rigid plastic piece covered on one or both sides by a flexible membrane. The membrane pressed against housing 24 of cycler 20 forms a pumping and valving membrane. FIG. 2 illustrates that disposable cassette 42 includes fluid pump chambers 44 that operate with the pneumatic pump chambers located at housing 24 of cycler 20 and fluid valve chambers 46 that operate with the pneumatic valve chambers located at housing 24 of cycler 20.

FIGS. 1 and 2 illustrate that disposable set 40 includes a patient line 50 that extends from a patient line port of cassette 42 and terminates at a patient line connector 52. FIG. 1 illustrates that patient line connector 52 connects to a patient transfer set 54, which in turn connects to an indwelling catheter located in the peritoneal cavity of patient P. Disposable set 40 includes a drain line 56 that extends from a drain line port of cassette 42 and terminates at a drain line connector 58. FIG. 1 illustrates that drain line connector 58 connects removeably to a drain connector 218 of water purifier 210.

FIGS. 1 and 2 further illustrate that disposable set 40 includes a heater/mixing line 60 that extends from a heater/mixing line port of cassette 42 and terminates at a heater/mixing bag 62 discussed in more detail below. Disposable set 40 includes an upstream water line segment 64a that extends to a water inlet 66a of water accumulator 66. A downstream water line segment 64b extends from a water outlet 66b of water accumulator 66 to cassette 42. In the illustrated embodiment, upstream water line segment 64a begins at a water line connector 68 and is located upstream from water accumulator 66. FIG. 1 illustrates that water line connector 68 is removeably connected to a water outlet connector 228 of water purifier 210.

Water purifier 210 outputs water and possibly water suitable for peritoneal dialysis (“WFPD”). To ensure WFPD, however, a sterilizing grade filter 70a is placed upstream from a downstream sterilizing grade filter 70b, respectively. Filters 70a and 70b may be placed in water line segment 64a upstream of water accumulator 66. Sterilizing grade filters 70a and 70b may be pass-through filters that do not have a reject line. Pore sizes for filters 70a and 70b may, for example, be less than a micron, such as 0.1 or 0.2 micron. Suitable sterilizing grade filters 70a and 70b may be provided by the assignee of the present disclosure. In an embodiment, only one of upstream or downstream sterilizing grade filter 70a and 70b is needed to produce WFPD, nevertheless, two sterilizing grade filters 70a and 70b are provided in the illustrated embodiment for redundancy in case one fails.

FIG. 2 further illustrates that a last bag or sample line 72 may be provided that extends from a last bag or sample port of cassette 42. Last bag or sample line 72 terminates at a connector 74, which may be connected to a mating connector of a premixed last fill bag of dialysis fluid or to a sample bag or other sample collecting container. Last bag or sample line 72 and connector 74 may be used alternatively for a third type of concentrate if desired.

FIGS. 1 and 2 illustrate that disposable set 40 includes a first, e.g., glucose, concentrate line 76 extending from a first concentrate port of cassette 42 and terminates at a first, e.g., glucose, cassette concentrate connector 80a. A second, e.g., buffer, concentrate line 78 extends from a second concentrate port of cassette 42 and terminates at a second, e.g., buffer, cassette concentrate connector 82a.

FIG. 1 illustrates that a first concentrate container 84a holds a first, e.g., glucose, concentrate, which is pumped from container 84a through a container line 86 to a first container concentrate connector 80b, which mates with first cassette concentrate connector 80a. A second concentrate container 84b holds a second, e.g., buffer, concentrate, which is pumped from container 84b through a container line 88 to a second container concentrate connector 82b, which mates with second cassette concentrate connector 82a.

In an embodiment, to begin treatment, patient P loads cassette 42 into cycler and in a random or designated order (i) places heater/mixing bag 62 onto cycler 20, (ii) connects upstream water line segment 64a to water outlet connector 228 of water purifier 210, (iii) connects drain line 56 to drain connector 218 of water purifier 210, (iv) connects first cassette concentrate connector 80a to first container concentrate connector 80b, and (v) connects second cassette concentrate connector 82a to second container concentrate connector 82b. At this point, patient connector 52 is still capped. Once fresh dialysis fluid is prepared and verified, patient line 50 is primed with fresh dialysis fluid, after which patient P may connect patient line connector 52 to transfer set 54 for treatment. Each of the above steps may be illustrated graphically at video monitor 32 and/or be provided via voice guidance from speakers 34.

For disposable set 40, the rigid portion of cassette 42 may be made for example of a thermal olefin polymer of amorphous structure (“TOPAS”) cyclic olefin copolymer (“coc”). The flexible membranes of cassette 42 may be made for example of a copolyletser ether (“PCCE”) and may be of one or more layer. Any of the tubing or lines may be made for example of polyvinyl chloride (“PVC”). Any of the connectors may be made for example of acrylonitrile-butadiene-styrene (“ABS”, e.g., for self-registering connector 100 of heater/mixing bag or container 62 and/or for concentrate connectors 80a, 80b, 82a, 82b discussed below) or a thermoplastic elastomer, such as Hytrel® (e.g., for bag adapters 80b and 82b that connect to concentrate connectors 80a and 82a), acrylic (e.g., for drain line connector 58) or PVC (e.g., for water line connector water line connector 68). Any of the bags or containers, such as heater/mixing bag or container 62 discussed below, may be made of PVC. The materials for any of the above components may be changed over time.

Control unit 22 may be programmed to cause cycler 20 to perform one or more mixing action to help mix dialysis fluid properly and homogeneously for treatment. For example, any of fluid pump chambers 44 may be caused to withdraw into the pump chambers some amount of mixed fluid (e.g., made from one or both first and second concentrates 84a, 84b and WFPD) from heater/mixing bag 62 and send such mixture back to heater/mixing bag 62 and repeat this procedure multiple times (described herein as a mixing sequence or “waffling”). In particular, to perform a mixing sequence, control unit 22 in an embodiment causes cycler 20 to close all fluid valve chambers 46 at cassette 42 except for the fluid valve chamber 46 to heater/mixing line 60 and heater/mixing bag 62. Fluid pump chambers 44 are stroked sequentially and repeatedly (i) pulling a possibly unmixed fluid combination of WFPD and concentrates from heater/mixing bag 62 into the pump chambers, followed by (ii) pushing the mixed WFPD and concentrates from the pump chambers back to heater/mixing bag 62 and (iii) repeating (i) and (ii) at least one time. Control unit 22 may be programmed to stroke fluid pump chambers 44 together so that they both pull and push at the same time, or alternatingly so that one pump chamber 44 pulls from heater/mixing bag 62, while the other pump chamber 44 pushes to heater/mixing bag 62, creating turbulence in heater/mixing line 60.

By providing container or bag 62 operable with cassette 42 and heater/mixing line 60, the WFPD from accumulator 66 and concentrates from first and second concentrate containers 84a and 84b are already at least partially mixed before entering the container or bag. Even if cassette 42 is not provided, the WFPD and at least one concentrate will mix partially in heater/mixing line 60 prior to reaching the container or bag.

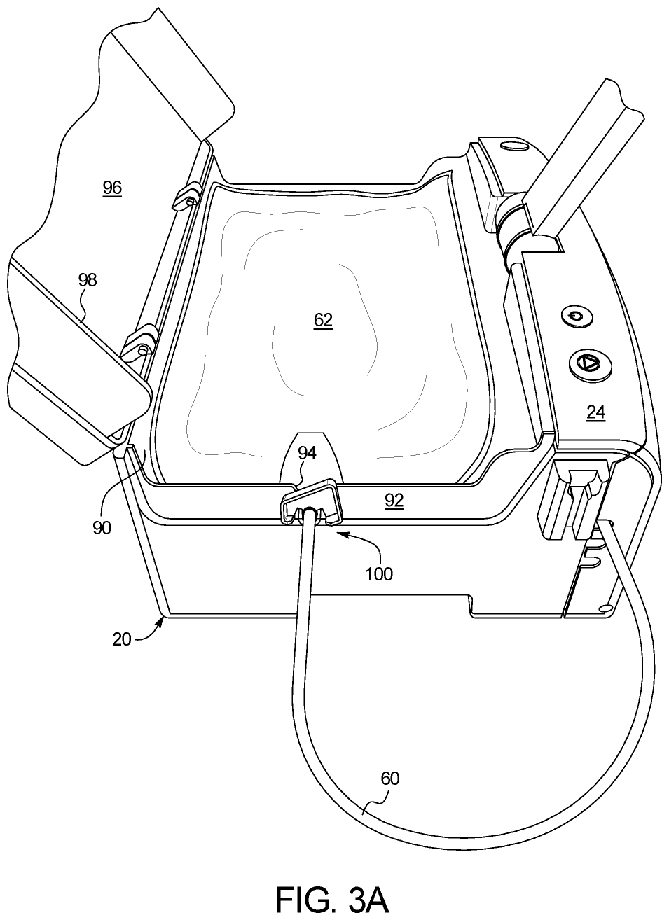

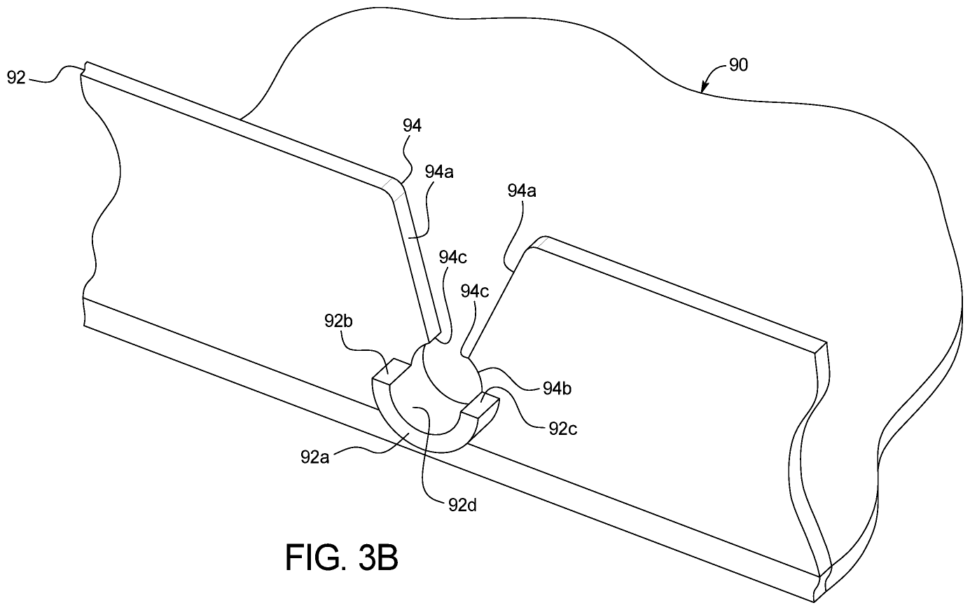

FIG. 3A illustrates a heating/mixing portion of housing 24 of cycler 20. Housing 24 includes a heater/mixing tray 90 located at the top of housing 24 for receiving heater/mixing bag 62. The heater of cycler 20, under control of control unit 22, is located beneath heater/mixing tray 90 and in one embodiment includes heating elements that contact heater/mixing tray 90. Heater/mixing tray 90 includes plural sidewalls, including sidewall 92 that defines a slot 94 for receiving a self registering heater/mixing bag connector 100 described in detail below. Housing 24 also includes a lid 96 connected hingedly to the back of housing 24 and at the top of heater heater/mixing tray 90. Lid 96 may be hinged open to locate and remove heater/mixing bag 62 and hinged closed onto housing 24 for insulation during heating. Lid 96 includes a sidewall 98 that closes just outside of sidewall 92 as described in more detail below. Lid 96 and sidewall 92 of housing 24 may be made of metal or plastic, while heater/mixing tray 90 is made of metal, such as aluminum, for conducting and withstanding heat from the fluid heater.

FIG. 3B illustrates the pertinent section of sidewall 92, including slot 94 in more detail. A sectioned semicircular flange 92a extends from sidewall 92. Semicircular flange 92a may be formed with or welded to sidewall 92. Semicircular flange 92a includes two upper horizontal surfaces 92b and 92c, which abut and help heater/mixing bag connector 100 to self-register as discussed below, to ensure that the port of connector 100 extending into heater/mixing tray 90 does so horizontally and roughly parallel with the bottom of heater/mixing tray 90.

Slot 94 in the illustrated embodiment includes an introductory V-shaped section 94a, which extends to a resting circular section 94b. A pinch point 94c separating V-shaped section 94a and circular section 94b is smaller than the contacting diameter of heater/mixing bag connector 100 in one embodiment. Patient P or other user accordingly feels a tactile “snap” when installing heater/mixing bag connector 100 into resting circular section 94b, indicating a proper installation. Pinch point 94c also tends to hold heater/mixing bag connector 100 in place, preventing the port from translating upwardly within slot 94, e.g., while heater/mixing bag 62 is being filled.

### Self-Registering Fluid Connector

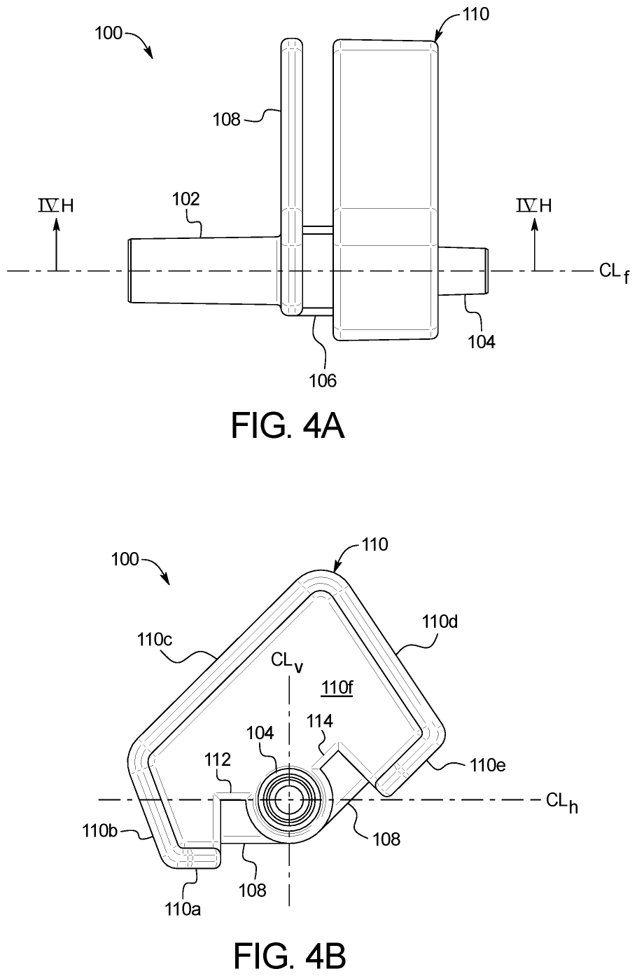

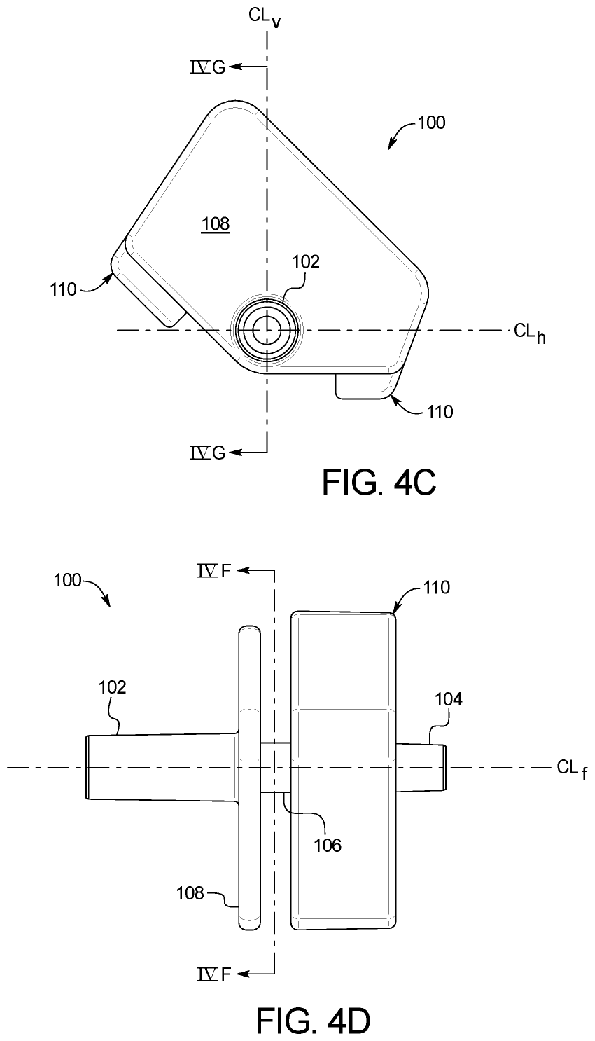

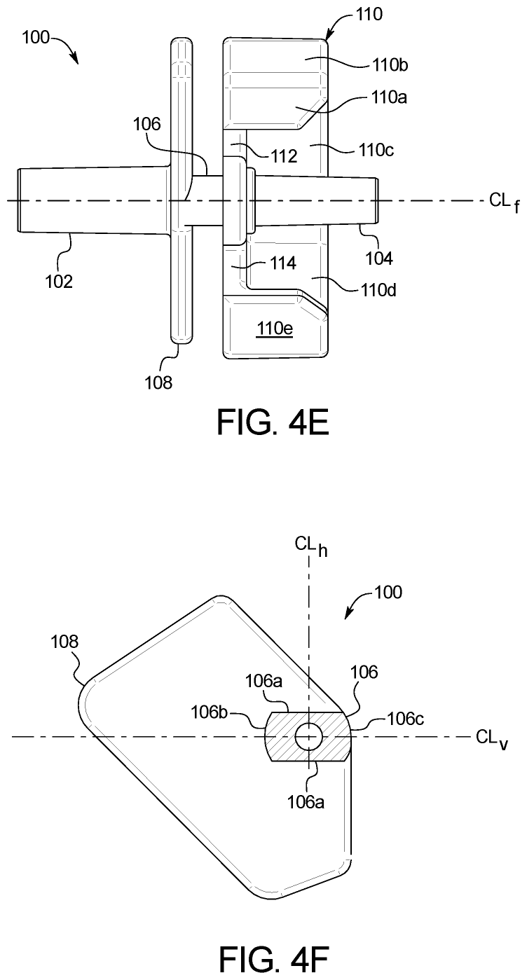

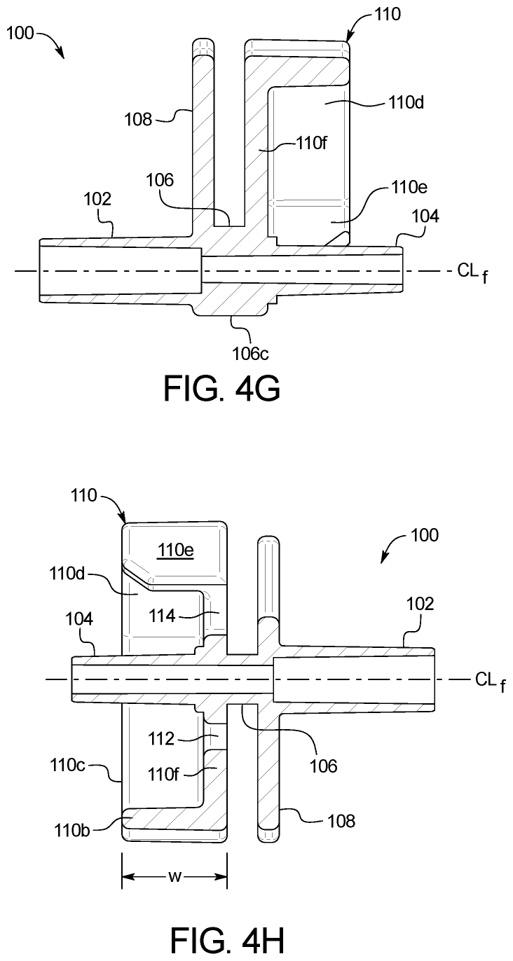

Referring now to FIGS. 4A to 4H, an example self-registering connector 100 is illustrated. As discussed above, connector 100 may be molded, e.g., injection molded, from acrylonitrile-butadiene-styrene (“ABS”). Connector 100 may be molded as a single piece or be assembled from separate molded pieces. FIG. 4A illustrates that connector 100 includes a first port 102 that extends into heater heater/mixing tray 90 and is sealed to heater/mixing bag 62, e.g., sealed via any combination of mechanical sealing, heat sealing, solvent bonding and/or ultrasonic welding. Connector 100 includes a second port 104 that extends outwardly from heater heater/mixing tray 90 and is sealed to heater/mixing line 60, e.g., sealed via any combination of mechanical sealing, heat sealing, solvent bonding and/or ultrasonic welding.

A pivot 106 is located between first port 102 and second port 104. The elevational side view of FIG. 4A illustrates that pivot 106 is longer along its side than is pivot 106 along its top in the top plan view of FIG. 4D or along its bottom in the bottom plan view of FIG. 4E. The sectioned view of FIG. 4F illustrates that pivot 106 is generally rectangular having longer sides 106a and a shorter top 106b and bottom 106c, but wherein top 106b and bottom 106c are rounded. Bottom 106c is in rotational contact with the rounded surface 92d of semicircular flange 92a extending from sidewall 92 of heater/mixing tray 90 when connector 100 is placed into slot 94. FIG. 4G illustrates that bottom surface 106c of pivot 106 in one embodiment may extend at least partially through a guide member 110, e.g., through a member wall 110f of guide member 110, discussed in detail below.

The side view of FIG. 4A, the top view of FIG. 4D, the bottom view of FIG. 4E and the sectioned views of FIGS. 4G and 4H illustrate that a guide wall 108 is in the illustrated embodiment a planar wall and resides between first or heater bag port 102 and pivot 106. FIGS. 4C and 4F illustrate that guide wall 108 from an inside-out (of heater/mixing tray 90) end view is generally a five-sided polygon with rounded corners. An inner surface of guide wall 108 rotates against the inner surface of sidewall 92 of heater/mixing tray 90 when connector 100 is placed into slot 94. Guide wall 108 also helps to prevent heater bag port 102 from pivoting upwards about a horizontal centerline CLₕ illustrated in FIGS. 4B, 4C and 4F, which in turn helps to introduce fluids to be mixed in heater bag 62, which may have different densities, in a horizontal manner that minimizes separation of the fluids. FIGS. 4B, 4C and 4F also illustrate that the contact between guide wall 108 and sidewall 92 of heater/mixing tray 90 also helps to prevent heater bag port 102 from pivoting sideways about a vertical centerline CLᵥ.

FIGS. 4A, 4D, 4E, 4G and 4H illustrate that connector 100 includes a guide member 110 that resides between second or heater line port 104 and pivot 106. FIGS. 4B and 4C illustrate that guide member 110 in general has the same five-sided shape as guide wall 108, although FIG. 4B in particular illustrates that guide member 110 extends further in certain directions than does guide wall 108.

FIGS. 4B, 4E, 4G and 4H illustrate that guide member 110 is in one embodiment not planar and instead has member flanges 110a to 110e that extend from a member wall 110f. Member flanges 110a to 110e and member wall 110f form a six-sided molded solid structure in the illustrated embodiment, which provides both the surface area and the strength to receive lid 96 of housing 24. In particular, the width w of member flange 110c (FIG. 4H) is large enough to provide ample room for any misalignment between sidewall 98 of lid 96 and sidewall 92 of heater/mixing tray 90. Member flanges 110a to 110e also add strength and rigidity to member wall 110f, so that the member wall does not bend or crack when lid 96 contacts connector 100.

FIG. 4B illustrates the position of connector 100 when it has been placed initially within slot, and heater/mixing bag 62 has been placed onto heater/mixing tray 90. In the illustrated position, member flanges 110c and 110e (which may be parallel to each other) may be angled with respect to horizontal centerline CLₕ at an angle from 30° to 60°, and in one embodiment at an angle of 45°. Member flange 110d may be angled with respect to vertical centerline CLᵥ at an angle from 15° to 50°, and in one embodiment at an angle of about 34°. Member flange 110a may be angled with respect to vertical centerline CLᵥ at an angle from 10° to 30°, and in one embodiment at an angle of about 20°. The matching sides of guide wall 108 to member flanges 110a to 110e may have correspondingly the same angles.

An inner surface of member wall 110f rotates against the outer surface of sidewall 92 of heater/mixing tray 90 when connector 100 is placed into slot 94. Member wall 110f helps to prevent heater bag ports 102 and 104 from pivoting about a horizontal centerline CLₕ illustrated in FIGS. 4B, 4C and 4F, which helps to introduce fluids to be mixed in heater bag 62 in a manner that minimizes separation of the fluids. FIGS. 4B, 4C and 4F also illustrate that the contact between member wall 110f and sidewall 92 of heater/mixing tray 90 also helps to prevent heater bag ports 102 and 104 from pivoting sideways about a vertical centerline CLᵥ.

FIG. 4B best illustrates that member wall 110f at its bottom (which does not have a member flange) forms first and second notches 112 and 114. The surfaces of notches 112 and 114 located directly adjacent to heater line port 104 in one embodiment each serve as stops. The surface of first notch 112 serves as a stop when connector 100 and heater bag 62 are initially loaded into slot 94 and heater/mixing tray 90, respectively. The surface of second notch 114 serves as a stop when lid 96 is closed and its sidewall 98 contacts member flange 110c of connector 100, causing connector 100 to rotate clockwise in FIG. 4B about fluid flow centerline CL_{f} illustrated in FIGS. 4A, 4D, 4E, 4G and 4H. In particular, the surface of first notch 112 abuts up against horizontal surface 92b of semicircular flange 92a when connector 100 is initially placed within slot 94 prior to rotation of connector 100, while the surface of second notch 114 abuts up against horizontal surface 92c of semicircular flange 92a to stop the rotation of connector 100.

In the above manner, connector 100 registers itself in a desired position for operation, especially when delivering multiple fluids to heater/mixing bag 62 to be mixed therein. The clamping via lid sidewall 98 of the surface of second notch 114 up against horizontal surface 92c of semicircular flange 92a of heater/mixing tray 90 locks the position of ports 102 and 104 into a desired position, e.g., a horizontal position, and holds the ports in such position even if connector 100 is subjected to fluid spikes or if cycler housing 24 is bumped, which could otherwise change the pitch or direction of ports 102 and 104 adversely from a fluid mixing standpoint.

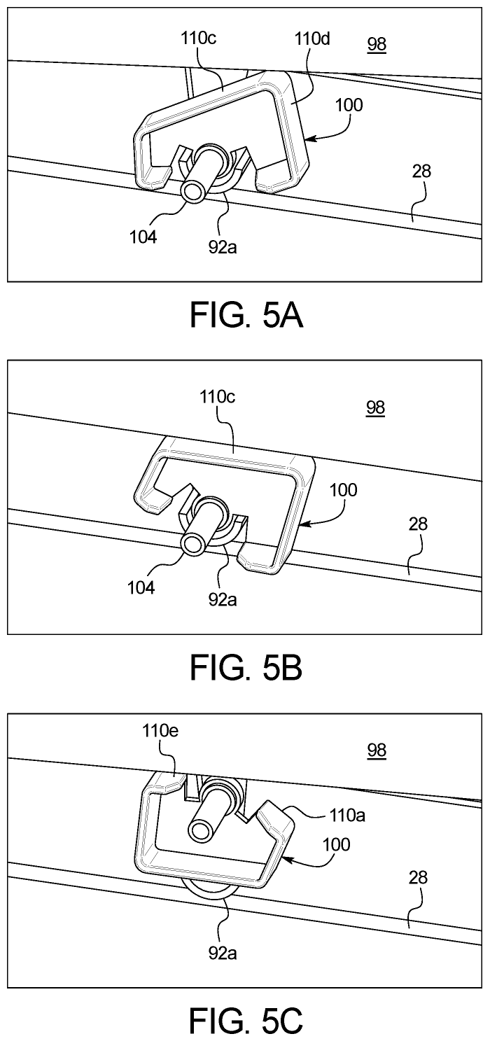

FIG. 5A illustrates from an outside-in view of heater/mixing tray 90, the situation in which sidewall 98 of lid 96 of housing 24 has been closed enough that it is just about to contact connector 100. As illustrated, connector 100 is in or is close to being in the position illustrated in FIG. 4B, such that the surface of first notch 112 abuts up against or is closed to abutting up against horizontal surface 92b of semicircular flange 92a. FIG. 4B and FIG. 5A also illustrate that the rounded interface between member flanges 110c and 110d is the first portion of connector 100 to be contacted by sidewall 98.

FIG. 5B illustrates from the outside-in view of heater/mixing tray 90, the situation in which sidewall 98 of lid 96 of housing 24 has been fully closed, such that it has contacted and rotated connector 100 clockwise in FIG. 5B. The rotation has caused member flange 110c of connector 100 to reside horizontally or substantially horizontally as illustrated. The surface of second notch 114 now abuts up against or is close to abutting up against horizontal surface 92c of semicircular flange 92a. The rotation of connector 100 between FIGS. 5A and 5B occurs as rounded bottom 106c of pivot 106 rotates against rounded surface 92d of semicircular flange 92a.

FIG. 5B also illustrates that housing 24 of cycler 20 may form a ledge 28 below semicircular flange 92a of sidewall 92 of heater/mixing 90. In the illustrated embodiment, connector 100 and in particular member flange 110d may be sized such that member flange 110e abuts against ledge 28 when sidewall 98 and lid 96 have been fully closed. In this manner, connector 100 is additionally trapped between the bottom of sidewall 98 and ledge 28 during operation, ensuring that connector 100 is maintained in the self-registered and desired position.

FIG. 5C illustrates from an outside-in view of heater/mixing tray 90, that an added benefit of connector 100 is that if the patient or caregiver attempts to load heater/mixing bag 62 upside down mistakenly, member flange 110c will abut against horizontal surfaces 92b and 92c of semicircular flange 92a, not allowing connector 100 to slide far enough into slot 94 to allow lid 96 to close fully. Thus if the patient or caregiver attempts to load heater/mixing bag 62 upside down, the tactile or visual failure of connector 100 to be inserted properly into slot 94 may alert the patient or caregiver to correct the mistake. Or, the visual failure of lid 96 to close fully may alert the patient or caregiver to correct the mistake. Additionally, it is contemplated to provide a switch, such as a normally electrically open limit switch, that closes electrically when lid 96 is fully closed, and to output such switch to control unit 22, which alarms when the patient or caregiver attempts to begin treatment and lid 96 is not fully closed. The alarm may be provided with an audio, visual or audiovisual message at user interface 30, prompting the patient or caregiver to check the placement of heater/mixing bag 62.

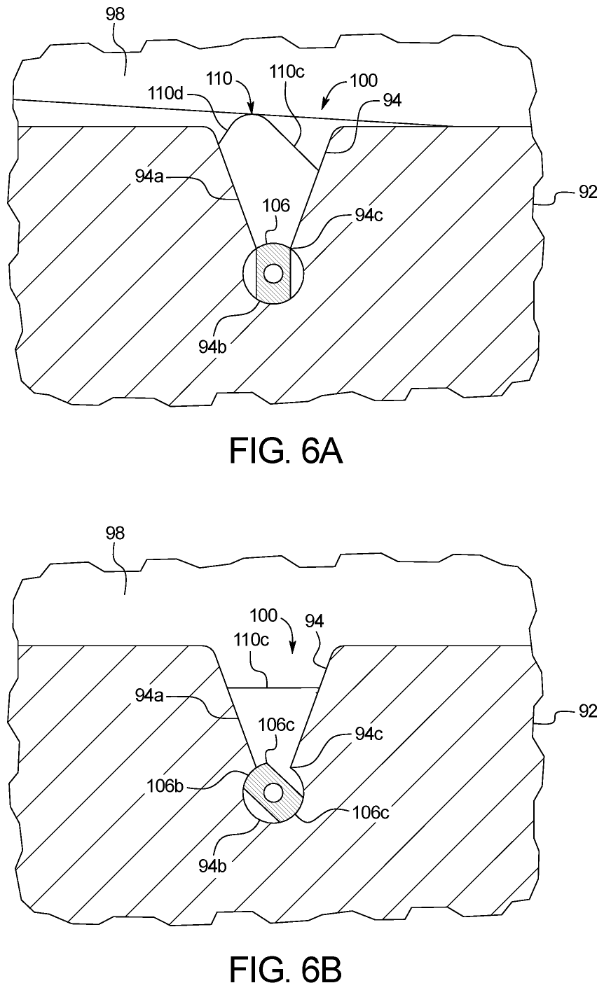

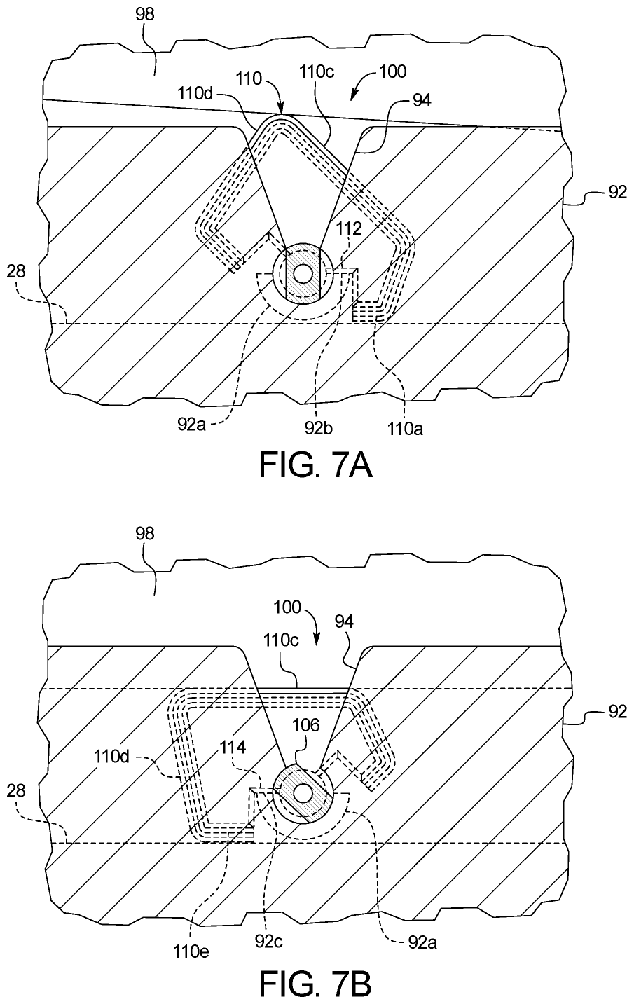

FIGS. 6A to 7B illustrate from an inside-out view of heater/mixing tray 90 with guide wall 108 having been cut away. Because the view in FIGS. 6A and 7A is from the inside-out, the hinge of lid 96 resides at the right side of the image as opposed to the left side in FIGS. 5A to 5C (outside-in view). FIG. 6A illustrates guide member 110 and in particular member flanges 110c and 110d of guide member 110 located behind sidewall 92 of heater/mixing tray 90. Connector 100 is in a totally non-rotated orientation with sidewall 98 of lid 96 just touching an interface between member flanges 110c and 110d of guide member 110. In this position, pivot 106 resides in a substantially vertical position in the illustrated embodiment. FIG. 6A also illustrates that the width of pivot 106 is made small enough to just slip through pinch points 94c separating V-shaped section 94a and circular section 94b of slot 94.

FIG. 6B illustrates connector 100 after it has been rotated into a fully rotated orientation with sidewall 98 of lid 96 resting horizontally across the top of member flange 110c. Pivot 106 correspondingly rotates the same amount as member flange 110c, which in an embodiment may be about 45° counterclockwise in the inside-out view of FIGS. 6A and 6B. FIG. 6B also illustrates that when connector 100 is rotated into its fully rotated position, rounded top 106b and bottom 106c of pivot 106 are rotated into communication with circular section 94b of slot 94, which further helps to secure connector 100 in place when in the fully rotated position. In an embodiment rounded top 106b and bottom 106c of pivot 106 have a slightly smaller radius than does circular section 94b of slot 94.

FIGS. 7A and 7B illustrate connector 100 in the same positions as FIGS. 6A and 6B, respectively, but show connector 100 in hidden lines behind sidewall 98 of lid 96. FIG. 7A further illustrates that in the totally non-rotated orientation with sidewall 98 of lid 96 just touching an interface between member flanges 110c and 110d of guide member 110, member flange 110a may contact ledge 28 of cycler housing 24, while first notch 112 contacts horizontal surface 92b of semicircular flange 92a. FIG. 7B further illustrates that in the fully rotated orientation with sidewall 98 of lid 96 lying horizontally on top of member flange 110c of guide member 110, member flange 110e contacts ledge 28 of cycler housing 24 as described above, while second notch 114 contacts horizontal surface 92c of semicircular flange 92a.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims. For example, connector 100 may be configured alternatively or additionally to translate into the desired position for treatment via the closing of lid 96.

The invention is claimed as follows:

1. A medical fluid connector comprising:
a first port;
a second port;
a pivot located between the first port and the second port;
a guide wall located between the first port and the pivot, the guide wall positioned to reside adjacent to a first side of a wall of a structure supporting the connector when inserted into the structure; and
a guide member located between the second port and the pivot, the guide member including a member wall positioned to reside adjacent to a second side of the wall of the structure supporting the connector when inserted into the structure, the guide member further including at least one member flange extending from the member wall, the at least one member flange sized so as to provide room for a lid of the structure to close onto the at least one member flange and rotate the connector about the pivot into a desired operating position.

2. The medical fluid connector of claim 1, wherein the guide wall and the member wall are at least partially shaped the same.

3. The medical fluid connector of claim 1, wherein the guide wall and the member wall are at least partially sized the same.

4. The medical fluid connector of claim 1, wherein the member wall is formed as a polygon, and wherein plural member flanges extend around a perimeter of the polygon, leaving an opening without a member flange at a bottom of the member wall, wherein the bottom of the member wall is configured to be received by the structure supporting the connector.

5. The medical fluid connector of claim 1, wherein the plural flanges and the member wall are molded together to form the connector.

6. The medical fluid connector of claim 1, wherein a bottom of the member wall forms a notch, which when the connector is rotated into the desired operating position, the notch abuts against a portion of the structure.

7. The medical fluid connector of claim 6, wherein the notch is a first notch, and wherein the bottom of the member wall forms a second notch that contacts a second portion of the structure prior to the connector being rotated.

8. The medical fluid connector of claim 1, wherein the pivot is formed in a rectangular shape, and wherein at least one of the sides is rounded for rotation against the structure supporting the connector.

9. The medical fluid connector of claim 1, wherein a bottom of the pivot extends through at least a portion of the guide member.

10. A dialysis system comprising:
a source of water made suitable for a dialysis treatment;
at least one concentrate for mixing with the water from the source;
a housing holding a dialysis fluid pump and forming a tray and a lid covering the tray; and
a disposable set operable with the dialysis fluid pump and in fluid communication with the source of water and the at least one concentrate, the disposable set including a container configured to accept the water and the at least one concentrate pumped by the dialysis fluid pump via a connector to mix for the dialysis treatment, wherein the connector is positioned and arranged such that when the container is placed onto the tray, the lid is able to close onto and move the connector into a desired operating position.

11. The dialysis system of claim 10, wherein the disposable set incudes a pumping cassette operable with the dialysis fluid pump, and wherein the connector includes a first port and a second port, the first port sealed to the container, and the second port sealed to a line leading from the connector to the pumping cassette.

12. The dialysis system of claim 10, wherein the lid is hinged to the housing and closes rotatingly onto the connector.

13. The dialysis system of claim 10, wherein the connector is configured to be rotated into the desired operating position when moved by the lid.

14. The dialysis system of claim 10, wherein the tray is a heater tray and the container is sized to enable the heater tray to heat the water and the at least one concentrate as they mix in the container.

15. The dialysis system of claim 10, wherein the tray defines a slot and includes a flange located beneath the slot, the flange including a surface, and wherein the connector is configured such that when moved into the desired operating position, the connector abuts against the surface.

16. The dialysis system of claim 15, wherein the surface is a first surface, wherein the flange includes a second surface, and wherein the connector is configured to contact the second surface prior to the connector being moved by the lid.

17. The dialysis system of claim 15, wherein the flange is a semicircular flange, and wherein the connector is configured to be rotated within the semicircular flange when the connector is moved by the lid.

18. The dialysis system of claim 10, wherein the housing further defines a ledge, and wherein the connector is sized and arranged to abut against the ledge when moved by the lid into the desired operating position.

19. A disposable set comprising:
a pumping cassette;
a heater/mixing container;
a line extending from the pumping cassette to the heater mixing container; and
a connector including
a first port sealed to the heater mixing container,
a second port sealed to the line extending from the pumping cassette,
a pivot located between the first port and the second port, and
a guide member located between the second port and the pivot, the guide member including a member wall positioned to reside adjacent to a side of a wall of a structure supporting the connector when inserted into the structure, the guide member further including at least one member flange extending from the member wall, the at least one member flange positioned and arranged to provide room for a lid of the structure to close onto the member flange and rotate the connector about the pivot into a desired operating position.

20. The disposable set of claim 19, which further includes a guide wall located between the first port and the pivot, the guide wall positioned to reside adjacent to an opposing side of the wall of the structure supporting the connector when inserted into the structure.

21. The disposable set of claim 19, wherein a bottom of the pivot extends through at least a portion of the guide member.

22. A dialysis method comprising:
providing a tray having a slot;
coupling a lid adjacent to the tray so that the lid may be opened and closed relative to the tray;
providing a fluid container having a connector sealed to the fluid container;
sizing the slot and a portion of the connector to fit into the slot when the container is placed onto the tray; and
configuring the connector such that when the lid is closed relative to the tray, the lid contacts the connector and moves the connector into a desired position for treatment.

23. The dialysis method of claim 22, wherein coupling the lid includes hinging the lid to the tray.

24. The dialysis method of claim 22, wherein moving the connector includes rotating the connector.

25. The dialysis method of claim 22, which includes configuring the slot, the connector and the lid so the lid continues to hold the connector in the desired position during treatment.

26. The dialysis method of claim 22, which includes configuring the connector and the slot such that a portion of the connector dead ends against a portion of the slot when the connector is in the desired position during treatment.

27. The dialysis method of claim 22, which includes providing the tray as part of a housing of a dialysis machine, and configuring the connector and the housing such that a portion of the connector dead ends against a portion of the housing when the connector is in the desired position during treatment.
